(19) United States
(12) Patent Application Publication
(10) Pub. No.: US 2017/0087013 A1
(43) Pub. Date: Mar. 30, 2017

(54) PHACOFRAGMENTATION AND PHACOASPIRATION TIP

(71) Applicant: Jose Luis Rodriguez Prats, Alicante (ES)

(72) Inventors: Jose Luis Rodriguez PRATS, Alicante (ES); Salvador Gabriel Delhom Muñoz, Provence of Valencia (ES)

(73) Assignee: Jose Luis Rodriguez PRATS, Alicante (ES)

(21) Appl. No.: 14/865,368

(22) Filed: Sep. 25, 2015

(51) Int. Cl. A61F 9/007; A61F 9/008

(52) U.S. Cl. CPC A61F 9/00736; A61F 9/00745; A61F 9/008; A61F 2009/0087; A61F 2009/00887; A61F 2230/0067; A61F 2230/0006; A61F 2230/0008

(57) ABSTRACT

Description of a phacofragmentation and phacoaspiration tip used in cataract surgery prepared for insertion of its distal end into any of the corneal incisions made for the fragmentation and aspiration of the lens. The tip is made up by an internally hollow cannular body that comprises a proximal portion for connection to any type of handle. The main characteristic is that this phaco tip presents a portion from the head portion up to the distal end and a distal end portion that in turn presents a pronounced external conical shape as it progresses towards the free end which length is considerably shorter than the intermediate portion, determining a gradual or staggered reduction in the thickness which, upon reaching the mentioned free end, forms a slimmed edge, polished although not cutting in itself, that ends in a blunt or rounded shape which is defined through an “r” radio.

### OBJECT OF THE INVENTION

This invention refers to a tip for phacofragmentation and phacoaspiration that offers essential features as far as innovation is concerned, along with outstanding advantages with respect to other known instruments known used for the same purposes in the current state of the art.

Specifically, the invention proposes the development of a phaco tip through an improved design to be used in cataract surgery by inserting it distal end through any of the corneal incisions made for fragmentation and aspiration in the opacified area of the crystalline lens (or as the case may be in transparent lenses) in cases of ultrasonic application as well as in cases of femtosecond laser beam by connection with the appropriate handle. An important peculiarity is that the tip of the phaco of the invention has a hollow cannular body, usually with a frustum-conical shape internally and externally, that in a distal end presents a sharper conical shape that determines the formation of an edge that diminishes towards the other end to finish in a blunt point, therefore in a moderate angle, i.e. not cutting in itself except during the aspiration process. For a better adaptation of the several situations existing in the current practice, the cannular body may be straight lengthways throughout its whole extension or it may present at the end one or more curved or angled parts with respect to the linear axis.

The field of application of the invention would be within the industrial sector committed in general terms to the design and fabrication of medical equipment for eye surgery.

### BACKGROUNDS

Cataract is a condition caused by a progressive opacification of the lens. A cataract surgery in summary consists of a surgical removal of the lens suffering from cataract and its subsequent replacement by an artificial intraocular lens.

All cataracts are not the same, which means that it is important to be aware that the opacified crystalline may have different degrees depending on the patient. Presently, the classification system most used to assess the cataract level is the LOCS III, a system that measures the opacity level of the crystalline in a scale that goes from 0 to 4.

The most usual techniques for cataracts surgery consist of an emulsification or fragmentation of the cataract in small pieces and their subsequent aspiration or suction. The most popular methods involve a fragmentation by application of ultrasounds, which is usually known as “phaco-emulsification” (or ultrasonic phaco-emulsification). There are though other advanced methods that carry out the fragmentation through the application of femtosecond laser (or picosecond), a technique that is known as “femtophaco” or “femtophacofragmentation”. Also know is the crystalline surgery through what is called the “phacorefractive” method.

Hereinafter is a brief explanation of each one of these methods, along with the associated advantages and inconveniences.

### Phacoemulsification:

This technique consists of opening two small incisions creating a main and a secondary port. The secondary port, of a size of approx. 1 mm, will be used for introducing the small supporting instruments (spatulas, hooks and/or injection tubes of viscoelastic material). The main port usually under 3 mm, specifically ranging from 1.8 to 2.8 mm, is used to introduce the end of the phacoemulsification device, commonly known as “phaco”. The phaco is a small manual instrument capable of fragmenting, irrigating and suctioning the pieces that includes in its distal end a small metallic tube called “tip” or “phacotip”, that fragments, emulsifies and absorbs the crystalline material. This metallic tube or cannula comes in a silicone case that has several lateral holes that allow the irrigation inside the eye (in order to keep the intraocular pressure equal to the suction generated). The ultrasound energy passes through this phaco tip in the form of high frequency beats, producing a pounding that in turn produces the breaking, fragmentation or emulsification of the cataract and also its suction.

In cases of bi-manual phacoemulsification both incisions, i.e. the main as well as the secondary one, are of a same size, presently of the order of approx. 1.4 mm. The tip of the phaco penetrates through one of the incisions while the other allows the entrance of the hydro-manipulator device.

In order to carry out the rupture and extraction operations, the phaco tip is connected to what is known as a phaco handle. This phaco handle is a device that includes a piezoelectric transductor that generates the ultrasound that makes the tip to vibrate. At the same time the phaco handle is connected to a console that includes a full equipment that allows the irrigation and later aspiration. The aspiration device uses a vacuum pump endowed with a mechanism that permits to change a good number of the values used, including the ultrasound energy. Physically, the phaco handle together with the ultrasound generator form a small instrument, of cylindrical design (similar to a felt-tip pen) of approx. 2 cm of diameter and 15 cm of length to which the tip is connected through a threaded joint to transmit the ultrasonic vibrations to the tip that emulsifies the crystalline. This handle in turn is connected to the console by means of an electric cable that permits to control the piezoelectric parameters as well as the entrance of the connection for irrigation and the exit of the aspiration connection.

As pointed out above, the suction or aspiration is made through a vacuum pump. This pump creates a depression outside the eye that produces an aspiration effect (phaco aspiration). The level of such depression may be controlled and should not cause undesirable fluctuations that could imply a risk of emptying other parts of the eye that must not be touched as that could create serious problems. This is the reason why the eye cavity where the surgery is taking place must be irrigated in order to maintain the intraocular pressure.

The ultrasound generator will be in charge of producing an ultrasound frequency that sends a vibration to the tip of the phaco that “hits” the crystalline lens causing its breaking into small pieces which will be later on absorbed (phacoemulsification, phacofragmentation). The ultrasound generator produces three types of movements on the tip of the phaco: an axial, a torsional and a rotational movement. The “violence” or intensity of these movements may be adjusted by controlling the frequency and the range of the ultrasonic wave thus adapting the energy transmitted.

To avoid eventual damages, it is advisable to conduct the operation with as little transmission of energy as possible, as the ultrasound emission in itself, the same as other more complicated types of energy, may affect the ocular structures, especially the corneal endothelium producing a corneal decompensation that may require a corneal transplant. All of it, besides the damage itself caused by the hammering effect.

In some cases, the above actions come from two independent instruments; one for the irrigation which is made through the secondary channel and another for the fragmentation—aspiration that is made through the main channel.

In any event it is a known fact among the experts in the matter that the vibration of the tip of the phaco, in the case of the independent irrigation mentioned above, produces injuries in the boundaries of the incision of penetration due to the vibratory friction of high frequency between the tip and the eye tissue. On the other hand, in the event of using an irrigation device in the same instrument, the necessary adjustment between the instrument and the incision to avoid leaks from the irrigation creates on the tip an amount of heat coming from the friction that is transmitted to the incision.

### Phemtophaco or Phemtophacofragmentación

In this operation, prior to the penetration of the phaco in the chamber that houses the crystalline, such crystalline is broken into small pieces according to the size required by the action of the femtosecond laser beam. Later on, once the crystalline lens has been fragmented, the steps to follow are identical to those described above, with the only difference that the phaco (tip of the phaco and silicone case for irrigation) through which the aspiration and removal are made, requires less ultrasound energy because the crystalline is already fragmented. The irrigation can be made exactly as in the previous case, i.e. through the secondary channel, in which case the phaco takes care of the aspiration and eventual phacoemulsification through its tip. This technique is safer although it must be pointed out that due to a higher cost in the femtosecond laser requirement is not as popular as the other methods we describe.

Cataract surgeries are more difficult, imply further risks and may become more complicated the greater the amount of ultrasonic energy liberated inside the eye when the cataract is harder, this is to say when the cataracts is of a higher level. In fact, hard cataracts imply the need of a phacoemulsification transmitting quite a lot of energy to the tip of the phaco, along with a phacoaspiration with high levels of depression.

In the phacoaspiration, a hard cataract would require higher support of ultrasound equipment in order to finish the fragmentation already initiated with the femtosecond laser.

The document pertaining to International Patent N^{o} WO 97/49343 describes a good example of a needle of phacofragmentation used in cataract surgery which has been designed in such a way that it presents a continued cutting edge tip located in the inner part of the surface of the cylindrical structure of the needle. Such cutting edge has been attained by splitting the structure or body of the needle in angle and beveling the external surface of at least the frontal part of the needle. The idea with this approach is to improve some aspects concerning the phacoemulsification such as the cutting capacity, the accuracy and the skewing power. The tip of the needle may also include an internal beveled surface in the portion of attack of the cutting edge. The description sketches attached to this document, specifically FIG. 11, show the cutting action of the tip of the needle derived from the energy applied to the needle.

Although the needle referred to by document WO 97/49343 implied a contribution of considerable operational improvements with respect to what the state of the art was at that time of its design and development—a process not free from drawbacks—such as for example the aggression for the crystalline cortex due to the cutting beveled shape of the tip of attack, together with a limited penetration into the extension of the beveled portion and the “braking” when this beveled portion disappears to find the smooth and parallel wall of the cannula. An additional inconvenience would appear when the tip somehow rotates during its time of operation, the beveled portion remains in one side and the penetration into the corneal incision may cut or lacerate the patient's cornea, in such a way that the lateral position of the tip would tear the back capsule of the crystalline lens, all of which represents a serious problem the present invention intends to avoid by means of a tip design that in theory would allow to protect the above back capsule

In accordance with the above description it is clear that the current techniques of phacofragmentation and phacoaspiration associated to a cataract surgery are susceptible of improvement considering the practical inconveniences they currently present. Hence, the main intention when developing the present invention is the improvement of a phacofragmentation and phacoaspiration tip in such a way that permits to provide effective solutions that remedy, or at least to a certain extent mitigate such inconveniences. This aim has been fully accomplished with the phacofragmentation and phacoaspiration tip that will be the subject matter of the description that follows, which essential parts are detailed in the part that refers to the characteristics of claim 1 attached.

In essence, the tip proposed by the present invention, designed for use in cases with or without cataracts, consists of a cannula made up by an elongated longitudinal body, internally hollow, which may be connected and used together with an irrigation and/or aspiration system such as those existing in the present state of the art, as well as with any type of handle. The phacofragmentation and phacoaspiration tip of this invention has the peculiarity that the body of the cannula in its external and internal profile or contour is shaped in a substantially frustum-conical shape with two parts of different conicity. The first outermost part, specifically the part corresponding to the distal end, is much shorter than the second part that stretches towards the rest of the body and presents a higher conicity degree in comparison with the second part, in such a way that the distal end of the cannula finishes in an edge preferably (although not exclusively) beveled, specifically of slimmed down profile, of a substantially reduced thickness but of such a caliber that it may not be considered as a cutting edge, unless by an aspiration action. This is to say, the external edge finishes, as opposite to the current tips of the present state of the art, with a predetermined thickness in its wall and with a curved external contour as well as a predetermined radio.

In the meantime, the external conicity of the second portion (i.e., the longer portion) gradually increases—as far as the internal diameter is concerned—from the distal end up to the proximal end, in a more or less continuous and uniform manner, or it may also present staggered variations, for example as cylindrical portions of variable diameters.

With a design as the one described above, the phacofragmentation and phacoaspiration tip proposed by the invention may be used in cases of soft was well as hard cataracts, in such a way that in the first case a previous fragmentation of the cataract by application of ultrasound or femtosecond laser would not be required because the geometric characteristics the distal extreme has been endowed with are sufficient to produce the fragmentation by means of a phacoaspiration, while in the case of hard cataracts, the same structural characteristics of the end portion of the tip would allow to carry out the phacoemulsification operation with a considerable less amount of ultrasound energy (of an order of 35% less than those operations using the current technique), with the consequent energy saving.

It is also important to point out that when using the femtosecond technique, regardless the type of cataract, the tip of this invention would only require phacoaspiration and not phacoemulsification.

In agreement with the above, the tip of the present invention offers a number of important advantages with respect to the application of the current tips, some of which will be detailed hereinafter:
Due to smaller diameter of the distal end, the tip allows a better penetration into the anterior chamber of the eye through a corneal incision, in such a way that in case of using a bi-manual technique, the tip may penetrate by any of the two incisions previously mentioned;
The tip allows a better adaptation to the corneal incision producing less deformation and reducing the risk of burns produced by an excessive use of ultrasonic energy. To such purpose the tip may be made, as deemed appropriate, with circular or oval sections;
The tip may include an internal coating made of a flexible material, with very low elasticity, in order “to cushion” the contact between the phaco tip and the optical tissue, minimizing or neutralizing any risk of injury in the incision either by friction or overheating.
The tip offers a universal solution because, as said, when allowing a straight or beveled end, it may be used in any type of phaco surgery (conventional, coaxial, micro-Phaco™ and microMICS™, as well as with any type of phaco and irrigation-aspiration system).
The tip may be straight lengthways or include a curved or angled part, for ozil and other phaco varieties.
The phaco tip of the invention may be endowed in the proximal end with any type of head that allow its adaptation to the several phaco handles existing in the market nowadays, including the conventional tightening means incorporated in the handles.
As an additional advantage, the internal core or lumen of the cannular body may be marked with stripes to force the rotation movement of the fragments suctioned by the inner part of the tip, thus improving the result of the aspiration.

Thus, while the conventional tips of the present state of the art, given a specific frequency, act as hitting or striking means upon the nucleus of the crystalline to fragment it and afterwards suck it out (to which purpose they present a wall of wide tubes to cause as much “damage” as possible to afterwards aspire the fragments through the lumen, the tip of the present invention acts in a totally opposite way in comparison with the conventional systems, as although the same aim of fragmentation is intended, the end result is achieved with a totally different strategy as far as rupture and fragmentation are concerned.

As it may be understood, a phacofragmentation and phacoaspiration tip with the characteristics indicated, besides of being much less aggressive than the conventional tips thanks to the non-cutting design of the tip (with the exception of cases of aspiration), permits to substantially improve the effectiveness associated to cataracts surgery, regardless the type—hard or soft—all that coupled with the additional advantage of the tip having thinner walls in the distal portion which produces a certain warpage when using a torsional or rotational phaco.

### BRIEF DESCRIPTION OF THE DRAWINGS

The above as well as other characteristics of the invention regarding a preferential realization may be clearly seen on the detailed description that follows. Such description is offered only by way of illustrative example and is in no way limited to the attached sketches, where:

FIG. 1 is a schematic and simplified upper-side view, lengthways, divided into a fourth, of a phacofragmentation and phacoaspiration tip made in accordance with the basics of the present invention, together with a D1 detail, using a larger scale, of the distal portion of the phaco tip, and

FIG. 2 shows a view the same as that shown under FIG. 1 although this time referring to a version of realization of the tip of the present invention, together with a D2 detail, using a larger scale of the end portion and two cross-wise examples (section I and Section II) of the cannular body.

### DETAILED DESCRIPTION OF PREFERRED EMBODIMENT

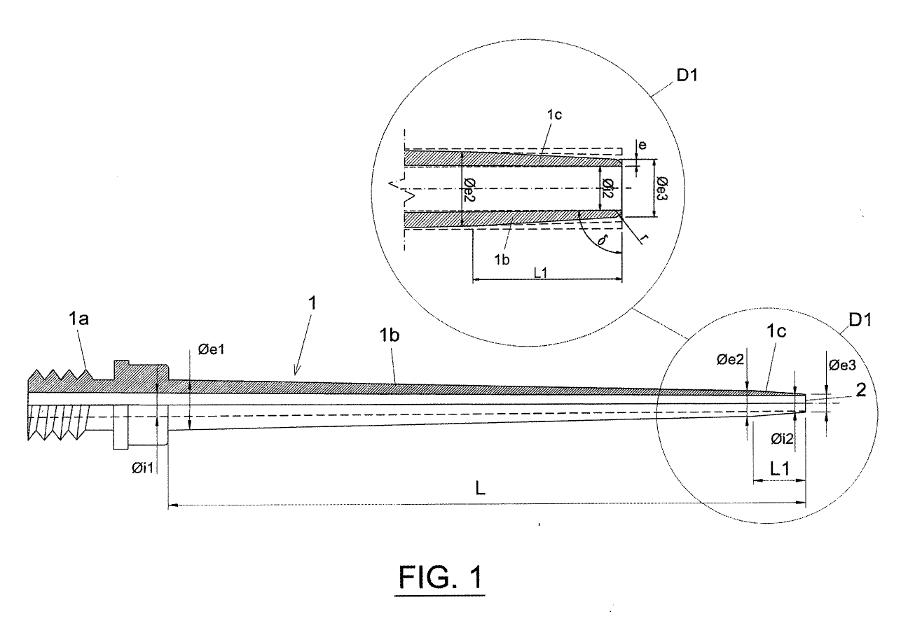

As indicated above, a detailed description of the suggested form of realization of the tip will be made below with the help of the attached drawings, where the same numerical references have been used to designate equal or similar parts. Thus, paying attention in the first place to the representation shown under FIG. 1, a schematic upper lateral view of the phacofragmentation and phacoaspiration tip is shown (phaco tip), with number 1, sectioned in a fourth and in lengthways direction following the tip of the phaco. In general terms, tip number 1 presents an elongated form and is internally hollow by virtue of an axial orifice that runs along its entire length. A head part may be seen in relation with the proximal end of the tip, that in the drawing is endowed, as a way of example, with a screw on the outer surface for connection or coupling to an appropriate handle, but that may include any other configuration among those known in the state of the art for connection to any kind of handle (not shown). After the head portion we have a cannular body identified with reference 1, which total length has been represented by “L”, that ends in a final distal portion—1c-which “L1” longitude represents only a minor portion of the total length “L”. The cannular body 1b has a frustum-conical form, in such a way at the beginning of the body, starting from the union to the head portion 1a, presents an external diameter indicated as Ø1e, and an internal diameter indicated as Ø1i, in such a way that the internal as well as the internal diameters are reduced towards the distal end.

Thus, in the realization suggested, the decrease of the external diameter is progressive until reaching the 1c distal end where the external diameter has been indicated as øe2. Starting from that point of transition, the degree of conicity increases so that the diameter of the body becomes narrower sooner in comparison with portion 1b until reaching the end where the external diameter has been represented as Øe3. In accordance with the above, we have the relation of: Øe1>Øe2>Øe3. The experts in the matter will understand that in an alternative form of realization, this progressive reduction towards the distal end of the tip may be staggered instead of gradual, without changing the functionality neither the effectiveness of the phacofragmentation and phacoaspiration tip of the invention.

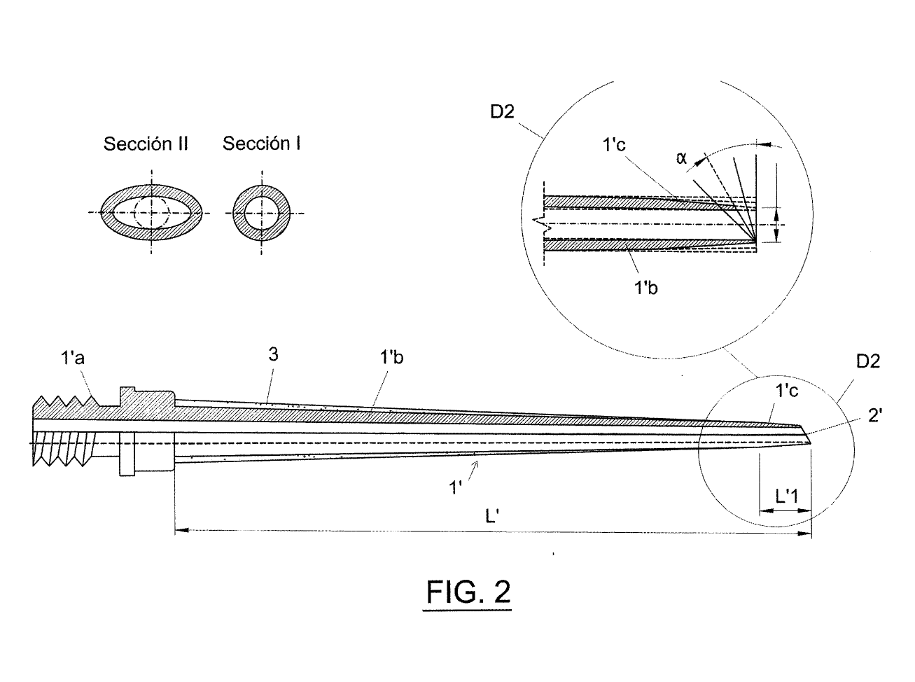

On the other hand and as said, the internal diameter is also reduced progressively when advancing towards the distal end, where it reaches the value represented by Øi2. In the form of realization shown under FIG. 2, this internal diameter reduction is continued and uniform although this is a fact that should not be construed as a limitation, as the reduction of the internal diameter, in other realizations, could be spaced out or staggered by sections. Therefore Øi1>Øi2 is fulfilled.

D1 illustrates a representation of the end portion 1c to higher scale. In this case, we may see the higher degree of conicity portion 1c has been endowed with in comparison with the longer intermediate portion. This higher conicity gives rise to a sharp and progressive reduction of the thickness of the wall of the cannular body, in such a way—and as clearly seen on D1—that the distal end indicated with reference 2 presents a polished or refined edge although not sufficiently sharp as to become cutting in itself.

The thickness of this polished edge has such a commitment value that without being sharp as explained above, is sufficient to fragment the cataract when carrying out an aspiration. In fact, the external arrangement of the outer edge of portion 1c (i.e. the attacking edge) is such that it shows the thickness indicated in the graphic representation as “e”, with an external profile visibly arched, which curvature radio has been represented as “r”. Such radio may be of variable magnitude. Preferably the above radio magnitude “r” should be within a range of values going from 0 to “e”.

On the other hand, the diameter of the internal lumen of the cannular body progressively increases as it advances from the distal end towards the head portion, which makes the aspiration of the fragments that penetrate by distal end 2 easier and free from any unwanted possibility of jamming as it avoid the “funnel” effect existing in other conventional tips.

As shown in FIG. 1, and better seen in D1, the profile or contour of the terminal edge of distal end 2 is straight in a cross section, this is to say, vertical to the longitudinal axis of the body, and therefore a perfect circle of internal diameter Øi2, as stated above

Now with reference to FIG. 2 of the drawings, we have a representation equivalent to FIG. 1, where the tip of the phaco indicated in general with reference 1′ has been divided lengthways in the same portions, namely a head portion 1′a, a lengthways portion of the cannular body 1′b of longitude “L”, and a distal end portion 1′c of longitude “L′1”. The phaco tip 1 is hollow internally throughout its entire length by virtue of a continued axial orifice that offers internal as well as external conical shape with an increase in the conicity degree in the end portion 1′, the same as described for tip 1 of FIG. 1. However, unlike the latter, the body of phaco tip 1 has been endowed with an external coating, indicated with reference number 3, designed with a reduction in the thickness also in the direction of the distal end made, as explained earlier on, with a flexible material such as elastomer which elasticity component is very low and that provides the “cushioning” intended, so that the contact between the tip of the phaco and the eye tissue is minimum, or even better, equal to zero thus avoiding injuries in the corneal incision either by friction or heating when applying ultrasonic energy in cases of fragmentation of hard cataracts that also operates as a seal.

Additionally, in this realization we may find characteristics that involve a beveled end. As it may be seen on D2, the outermost portion 1c of the distal end, seen in a cross section, presents a cut that, in a tilted view, includes an angle α with respect to the vertical. This α angle may be chosen depending on the function of the final application of the phaco tip 1. Of course it will be understood that the finishing characteristics of the blunted “attacking” edge of the phaco tip of this realization may be the same as those already discussed in connection with the realization of FIG. 1

Likewise, and going back to D1, the representations allow to appreciate another important feature of the phaco tip of the present invention that concerns the angle identified with reference “δ”, located between the longitudinal axis of the tip and the area that runs in vertical to the area of “attack”. This angle should preferably be greater than 90°, this is to say an obtuse angle for any value of α≧0.

Lastly, FIG. 2 also presents two sections, Section I and Section II. These representations refer to other possibilities of alternative realization as they show a cross section of the body in a circular form (Section I) and in oval form (section II). The choice between one or the other should be made by the professional depending on the specific requirements in order to allow an adaptation to the corneal incision producing as little deformation as possible and thus reducing the risk, as indicated above, of possible burns by excessive use of the ultrasonic energy

It is not considered necessary to extend the contents of this description any further for an expert in the matter to be able to understand its scope as well as its advantages and to finally carry out its practical realization.

Notwithstanding the above, and since the description only pertains to an example of suggested realization, it should be understood that multiple detail variations may be introduced in its essential parts, variations that are also protected and that may affect the form, the size or the fabrication materials of the whole instrument or any part thereof, without this implying any kind of alteration of the invention as a whole that should be restricted solely to the claims detailed hereunder:

1. Phacofragmentation and phacoaspiration tip for cataract surgery, regardless the cataract hardness, prepared for insertion of its distal end into any of the corneal incisions made for fragmentation and aspiration of an area—whether or not opacified—of the lens. This phaco tip (1;1′) has been conceived as a cannular body, hollow by virtue of an axial orifice extended throughout the length of the tip, endowed with a proximal portion (1a; 1′a) designed for connection with any type of handle. Its main characteristic is that the body of the phaco tip presents, from the head portion to the distal end (2;2′) an intermediate section (1b) of cannular body with a length (“L”) that spreads through most of the total length of the tip. After that intermediate portion (1b), the tip presents a distal end portion (1c; 1′c) that in turn presents a strong external conicity as it advances towards the free end, and a length (“L”; “L′1) a lot shorter than the length of the intermediate portion (1b; 1b′), which determines a reduction of the thickness that when reaching the mentioned free end (2;2′) forms a slimmed edge, sharp but not cutting in itself to end in a blunt point.

2. Tip as per claim 1, characterized in that the mentioned intermediate longer portion (1b) may also be endowed with external and internal conicity. The external conicity rates are such that the internal and external diameters are reduced when advancing in direction of the distal end, with both rates of conicity in the intermediate portion (1b) lower than in the distal end portion (1c; 1′c)

3. Tip as per claim 1 characterized in that the reduction of the internal diameter of the phacofragmentation and phacoaspiration tip (1;1′) going in direction of the final end may either be continued and uniform or staggered by sections.

4. Tip as per claim 1 characterized in that it may incorporate, as an option, an external coating (3) of a flexible material such as elastomer, i.e. a material of low elasticity in order to provide certain “cushioning” in the contact between the phaco tip (1;1′) and the eye tissue of the patient, thus minimizing or avoiding any risk of injury in the corneal incision.

5. Tip as per claim 1 characterized in that optionally the profile or contour of the end edge (2′) may be vertical to the length of the axis, or it may form an angle (α) with the vertical of variable size.

6. Tip as per claim 1, characterized in that the configuration of the transversal section of the phaco tip (1;1′) may be circular or oval.

7. Tip as per claim 1 characterized in that the distal end (2; 2′) that forms the edge of attack of the end portion (1c; 1′c) of the tip has a wall thickness (“e”) with an external curved profile in angle.

8. Tip as per claim 7 characterized in that the magnitude of the radio (“r”) of the curved profile of the distal end (2;2′) of the external portion (1c; 1′c) of the phaco tip ranges from values between “0” and “e”.

9. Tip as per claim 1, characterized in that the amplitude of the angle (δ) measured between the lengthways axis of the tip and the area vertical to the fronts of attack in the distal end (2; 2′) of the outermost end (1c; 1′c) of the phaco tip ranges between values equal to or higher than 90° for values of α≧0.
